(19) United States
(12) Patent Application Publication
(10) Pub. No.: US 2017/0347950 A1
(43) Pub. Date: Dec. 7, 2017

(54) SYSTEM AND METHOD FOR BREATH MONITORING MATTRESS

(71) Applicant: Gail Yvette Jones, District Heights, MD (US)

(72) Inventor: Gail Yvette Jones, District Heights, MD (US)

(21) Appl. No.: 15/445,555

(22) Filed: Feb. 28, 2017

Related U.S. Application Data
(60) Provisional application No. 62/345,762, filed on Jun. 4, 2016.

(51) Int. Cl. A61B 5/00; A47C 21/00; A47C 27/06; A47D 15/00; A47C 31/00; G08B 21/06; A61B 5/08

(52) U.S. Cl. CPC A61B 5/4818; G08B 21/06; A61B 5/6892; A61B 5/746; A61B 5/7455; A61B 5/742; A61B 5/7282; A47C 21/006; A61B 5/0816; A61B 5/0004; A47D 15/001; A47C 31/00; A47C 27/064; A61B 2503/04

(57) ABSTRACT

A new approach is proposed that contemplates system and method to support breath monitoring of a person via a plurality of breathing sensors embedded in a mattress the person is resting on. Once activated, each of the breathing sensors is configured to detect breathing activities or body movements caused by the breathing activities of a person lying on the mattress and report the breathing activities detected as electronic signals to a breathing monitor. Upon accepting the signals from the breathing sensors embedded in the mattress, the breathing monitor is configured to identify a breathing pattern based on the breathing activities of the person and detect any abnormal change in the breathing pattern of the person. If such abnormal change is detected, the breathing monitor is configured to generate and provide an alert to a monitoring party/caregiver of the person and/or produce an alarm to wake up the person in sleep.

### CROSS-REFERENCE TO RELATED APPLICATIONS

This application claims the benefit of U.S. Provisional Patent Application No. 62/345,762, filed Jun. 3, 2016, and entitled “Breath Rite Mattress,” which is incorporated herein in its entirety by reference.

### BACKGROUND

Sleeping disorder or any disorder that may cause a person to stop breathing during sleep affects well-being of millions of people. For a non-limiting example, sleep apnea is a common type of sleep disorder in which a person has one or more pauses in breathing or shallow breaths while sleeping, wherein the breathing pauses can last from a few seconds to minutes. More seriously, sudden infant death syndrome (SIDS) is the leading cause of death among infants under one year old. Although the exact cause of SIDS remains unclear, it may be reflected in an abnormal breathing pattern of an infant.

Wearable motion and/or breathing sensors have become available lately, which when attached to a person's body, clothes, or diaper (in the case of an infant), can monitor the person's body movements and/or breathing activities during sleep and provide information, indication, or alarm when an abnormal pattern in the person's body movement or breathing pattern is detected. Such wearable breathing sensors, however, are often not reliable due to random movements of the person during sleep, which may cause the sensors to get loose relative to the person's body or even trigger false alarm at times. In addition, it is often unrealistic to expect a person to consistently wear the sensors attached so close to his/her body, which can be intrusive especially for an infant. A better solution is desired, which would help to detect abnormal sleeping patterns of people especially infants without them being consciously aware of the presence of the sensors used for monitoring their body movements and/or breathing activities.

The foregoing examples of the related art and limitations related therewith are intended to be illustrative and not exclusive. Other limitations of the related art will become apparent upon a reading of the specification and a study of the drawings.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 depicts an example of a system diagram to support a breath monitoring mattress for sleeping disorder detection in accordance with some embodiments.

FIG. 2 depicts examples of various possible locations of breathing sensors in the mattress in accordance with some embodiments.

FIG. 3 depicts examples of various resting state of a person on the mattress in accordance with some embodiments.

FIG. 4 depicts an example of a flowchart of a process to support a breath monitoring mattress for sleeping disorder detection in accordance with some embodiments.

### DETAILED DESCRIPTION OF EMBODIMENTS

The following disclosure provides many different embodiments, or examples, for implementing different features of the subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

A new approach is proposed that contemplates system and method to support breath monitoring of a person via a plurality of breathing sensors embedded in a mattress the person is resting on. Once activated, each of the breathing sensors is configured to detect breathing activities and/or body movements caused by the breathing activities of the person lying on the mattress and report the breathing activities detected as electronic signals to a breathing monitor. Upon accepting the signals from the breathing sensors embedded in the mattress, the breathing monitor is configured to identify a breathing pattern based on the breathing activities of the person and detect any abnormal change in the breathing pattern of the person. If such abnormal change in the breathing pattern is detected, the breathing monitor is configured to generate and provide an alert to a monitoring party/caregiver of the person and/or produce an alarm to wake up the person in sleep.

By embedding the breathing sensors appropriately in the mattress the person sleeps on, instead of attaching the sensors to the person's body or clothes, the proposed approach enables more reliable collection of signals/data about the person's body movements and/or breathing activities that does not depend on how the person may position/attach the sensors and/or how the sensors may move with respect to the person's body when he/she moves during sleep. Such reliable detection of the person's breathing activities reduces chances of missed detection and/or any false alarms of breathing abnormality of the person. As a result, the party monitoring the person's breathing activities (e.g., parent of an infant or caregiver of the person) may get notified promptly and the person who is experiencing a sleep disorder may be woken up by an alarm promptly as soon as a breathing abnormality of the person is detected.

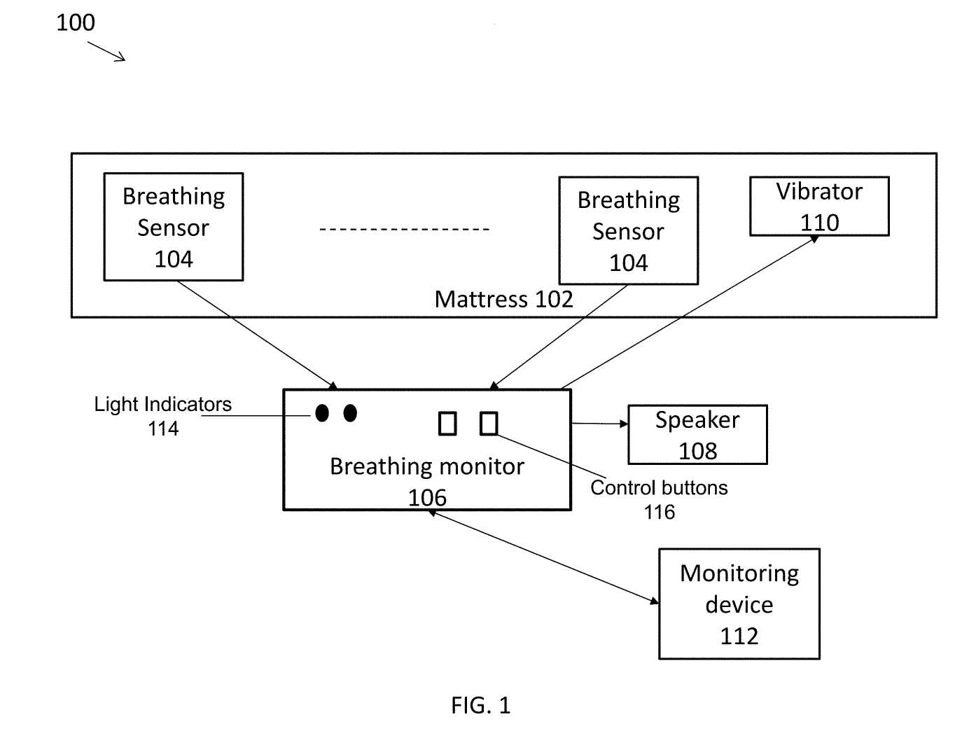

FIG. 1 depicts an example of a system diagram to support a breath monitoring mattress for sleeping disorder detection. Although the diagrams depict components as functionally separate, such depiction is merely for illustrative purposes. It will be apparent that the components portrayed in this figure can be arbitrarily combined or divided into separate software, firmware and/or hardware components. Furthermore, it will also be apparent that such components, regardless of how they are combined or divided, can execute on the same host or multiple hosts, and wherein the multiple hosts can be connected by one or more networks.

In the example of FIG. 1, the system 100 includes at least one or more breathing sensors 104 embedded in a mattress 102, and a breathing monitor 106 connected to the breathing sensors 104. Here, the mattress 102 can be of various types and/or sizes. For non-limiting examples, the mattress 102 can be but is not limited to, a crib-sized mattress for infant or a toddler-sized mattress for a toddler, wherein the system 100 is configured to prevent the infant/toddler sleeping on the mattress 102 from suffering SIDS and/or infant sleep apnea. In some embodiments, the mattress 102 can be in size of one of Twin, Full, Double, Queen, King, and California King, wherein the system 100 is configured to prevent an adult sleeping on the mattress 102 from sleep apnea or any other type of sleep disorder.

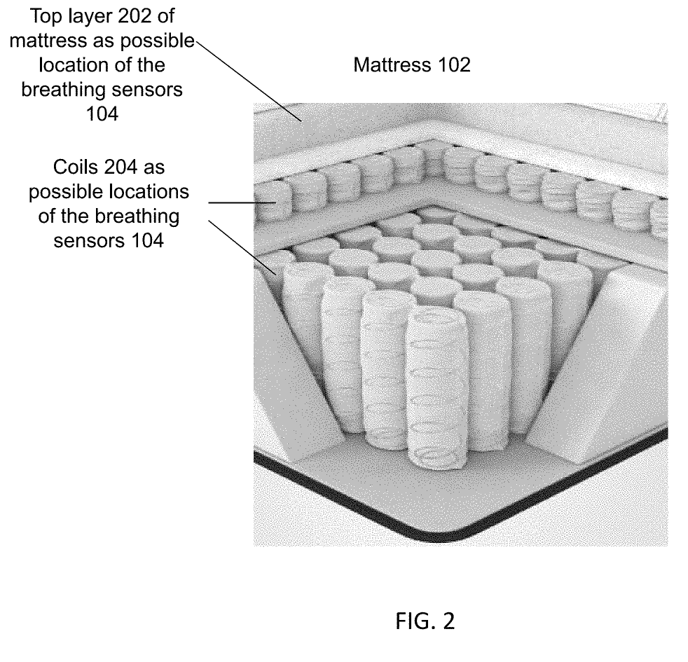

In the example of FIG. 1, the breathing sensors 104 are electronic sensing devices embedded in the mattress 102 and configured to detect the body movements and/or breathing activities of a person occupying and sleeping on the mattress 102. In some embodiments, as depicted by the example of FIG. 2, the breathing sensors 104 are positioned within or underneath a top layer 202 of the mattress 102 in proximity to the person who occupies the mattress 102 for accurate detection of his/her body movements and/or breathing activities. In some embodiments, the breathing sensors 104 are configured to detect any change or distortion in the shape or contour of the top layer of the mattress 102 caused by the body movements and/or breathing activities of the person and transmit a signal to the breathing monitor 106 when such change is detected.

In some embodiments, the breathing sensors 104 are coupled/attached to one or more coil springs 204 inside the mattress 102 as depicted by the example of FIG. 2. When the coil springs are suppressed as a result of the body movements and/or breathing activities of the person, the breathing sensors 104 are configured to detect such suppression and transmit a signal to the breathing monitor 106 accordingly.

In some embodiments, the breathing sensors 104 are configured to provide electronic data/signals of the person's body movements and/or breathing activities to the breathing monitor 106 via a wired connection between the breathing sensors 104 and the breathing monitor 106. In some embodiments, the breathing sensors 104 and the breathing monitor 106 are configured to communicate with each other via their respective communication interfaces (not shown) over a communication network following certain communication protocol, such as TCP/IP protocol. Here, the communication network can be but are not limited to, Internet, intranet, wide area network (WAN), local area network (LAN), wireless network, Bluetooth, WiFi, and mobile communication network. The physical connections of the network and the communication protocols are well known to those of skill in the art.

In the example of FIG. 1, the breathing monitor 106 is configured to accept and analyze signal received from the breathing sensors 104 about the person's body movements and/or breathing activities. Here, the breathing monitor 106 is an electronic device external to the mattress 102. In some embodiments, the breathing monitor 106 is a dedicated electronic hardware device that includes at least a microprocessor, a storage unit such as a non-volatile memory (also referred to as secondary memory) of the device for storing data and software instructions to be loaded into memory (also referred to as primary memory) and executed by the microprocessor for practicing one or more processes. In some embodiments, the microprocessor may be a digital signal processor formed of application specific integrated circuits for performing the processes.

In some embodiments, the breathing monitor 106 may further include a display screen and one or more input devices, control buttons, and LCD indicators (not shown). Here, the breathing monitor 106 can be either plugged into a power outlet via a cord or battery-powered.

In some embodiments, the breathing monitor 106 is a general purpose computing/communication device having a breath monitoring software (e.g., a breath monitoring app) running on it. Here, The computing/communication device can be but is not limited to a mobile phone (e.g., a smartphone) or a tablet such as an iPhone, an iPad, a Google's Android device, a laptop PC, or any mobile computing and/or communication device capable of running a software component or app for breath monitoring.

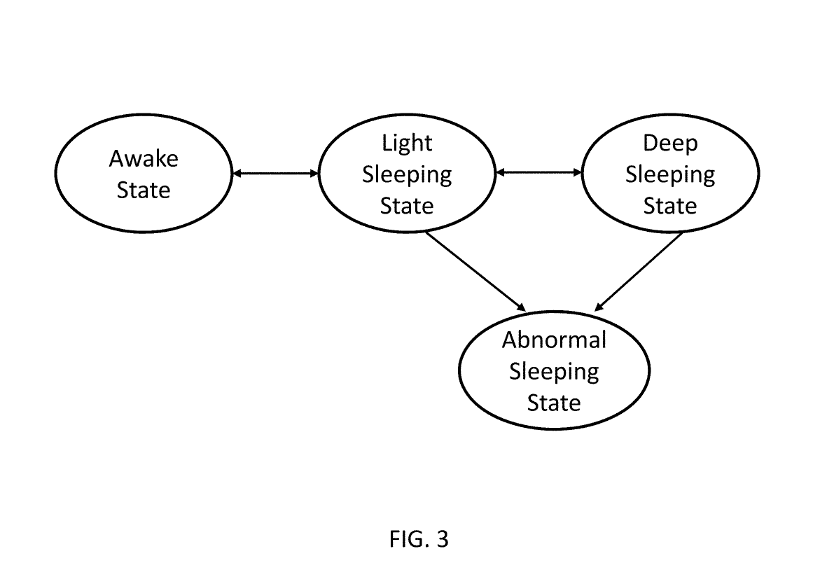

In some embodiments, the breathing monitor 106 is configured to identify a breathing pattern of a person lying on the mattress 102 based on analysis of data of the person's body movements and/or breathing activities collected by the breathing sensors 104. Here, the identified breathing pattern may include one or more of frequency of the person's breath, duration and depth of each breath. In some embodiments, the breathing pattern of the person is used by the breathing monitor 106 to determine the current resting state of the person on the mattress 102, where the resting state can be but is not limited to one of awake state, light sleeping state, and deep sleeping state as shown by the example of FIG. 3. For a non-limiting example, if the person is having slower, deeper, regular but less frequent breaths than when awake, he/she is most likely in a deep sleeping state, where the sleeping disorders are most likely to happen. Note that the transition among different resting states of the person, such as from awake to sleeping or from sleeping to awake usually happens gradually over time, not abruptly. In some embodiments, the breathing monitor 106 is configured to record/store the breathing pattern of the person in a storage unit (not shown) of the breathing sensors 104 for later detection of any sudden change in the person's breathing pattern.

In some embodiments, the breathing monitor 106 is configured to detect any sudden or abnormal change in the person's breathing pattern while the person is in a sleeping state, wherein such sudden or abnormal change may be an indication that the person is experiencing a sleeping disorder and entering an abnormal sleeping state. Here, the sudden or abnormal change may happen in one or more of the frequency, duration and depth of the person's breath. For a non-limiting example, the breathing monitor 106 is configured to detect that the person has stopped breathing if there is a sudden drop in the frequency of his/her breathe or the duration of his/her breathe becomes unusually long relative to the duration his/her normal breathe.

Once a sudden or abnormal change in the person's breathing pattern is detected, the breathing monitor 106 is configured to alert the person or his/her caregiver (e.g., parents of an infant or toddler or nurse of a patient) to correct the situation via various means. For non-limiting examples, in some embodiments, the breathing monitor 106 is configured to automatically trigger an alarm sound through a speaker 108 associated with or integrated within either the breathing monitor 106 or the mattress 102 to wake the person up. In some embodiments, the breathing monitor 106 is configured to automatically trigger a vibrator 110 embedded in the top layer of the mattress 102 to wake the person up. In some embodiments, the breathing monitor 106 is configured to turn on one or more LCD/LED indicators 114 on the breathing monitor 106 to alert a caregiver that a sudden or abnormal change in the person's breathing pattern has happened. In some embodiments, the breathing monitor 106 includes one or more control buttons 116 configured to enable a caregiver of the person to manually control operations and functionalities of the breathing monitor 106 such as turning on/off the alarm, light indicator, and/or the vibrator.

In some embodiments, the breathing monitor 106 is configured to provide information about the person's breathing pattern as well as the sudden or abnormal change detected in the breathing pattern to monitoring device 112 associated with a caregiver (e.g., parents of a baby or nurse of a patient at another room) and configured to receive such information when the caregiver is at a separate location from the breathing monitor 106 and/or the person sleeping on the mattress 102. Here, the monitoring device 112 can either be a dedicated monitoring device (e.g., baby monitor) or a general purpose computing device such as a mobile phone or a tablet or any computing device capable of running a software component or app for breath monitoring. In some embodiments, the breathing monitor 106 is configured to communicate with the monitoring device 112 wirelessly over a communication network such as Bluetooth and/or WiFi network.

In some embodiments, the monitoring device 112 is configured to present the information about the person's breathing pattern to the caregiver via a display. In some embodiments, the monitoring device 112 is configured to notify or alert the caregiver by sound or vibration when the sudden or abnormal change in the breathing pattern of the person is detected, e.g., when the person has stopped breathing. In some embodiments, the monitoring device 112 is configured to be pre-programmed to make emergency phone calls to 911 when the sudden or abnormal change in the breathing pattern of the person is detected if the caregiver is not present. In some embodiments, the monitoring device 112 is configured to provide the caregiver with instructions on how to deal with the person experiencing the sudden or abnormal change in his/her breathing pattern, e.g., how to perform CPR on the person experiencing a sleeping disorder. In some embodiments, the monitoring device 112 is configured to accept an instruction from the caregiver via an input interface (e.g., a keyboard or touchscreen) and take actions accordingly, e.g., playing an alarm sound via speaker 108 or creating a vibration at the mattress 102 via vibrator 110.

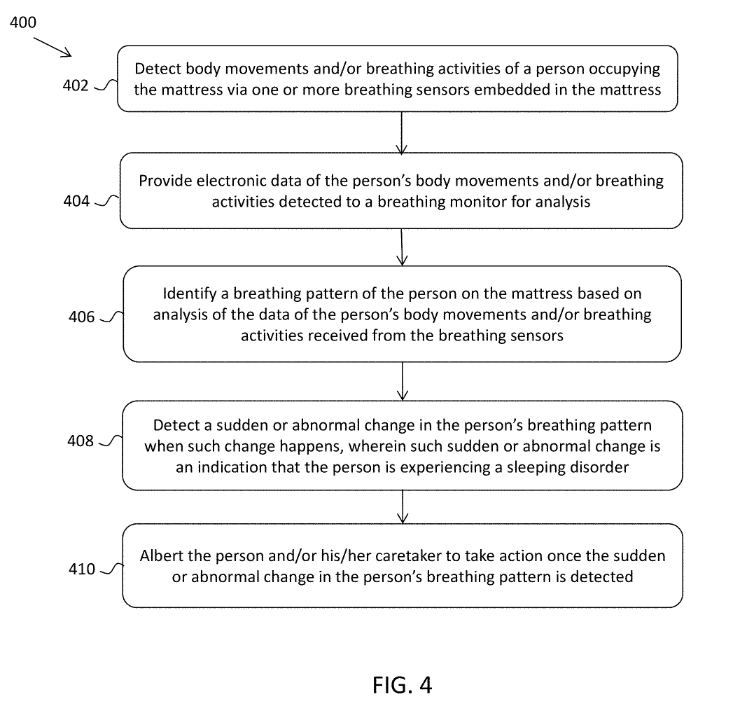

FIG. 4 depicts an example of a flowchart of a process to support a breath monitoring mattress for sleeping disorder detection. Although this figure depicts functional steps in a particular order for purposes of illustration, the process is not limited to any particular order or arrangement of steps. One skilled in the relevant art will appreciate that the various steps portrayed in this figure could be omitted, rearranged, combined and/or adapted in various ways.

In the example of FIG. 4, the flowchart 400 starts at block 402, where body movements and/or breathing activities of a person occupying or sleeping on the mattress are detected via one or more breathing sensors embedded in the mattress. The flowchart 400 continues to block 404, where electronic data/signals of the person's body movements and/or breathing activities detected by the breathing sensors is provided to a breathing monitor for analysis. The flowchart 400 continues to block 406, where a breathing pattern of the person on the mattress is identified based on analysis of the data of the person's body movements and/or breathing activities received from the breathing sensors. The flowchart 400 continues to block 408, where a sudden or abnormal change in the person's breathing pattern is detected when such change happens, wherein such sudden or abnormal change is an indication that the person is experiencing a sleeping disorder. The flowchart 400 ends at block 410, where the person and/or his/her caregiver is alerted to take action once the sudden or abnormal change in the person's breathing pattern is detected.

One embodiment may be implemented using a conventional general purpose or a specialized digital computer or microprocessor(s) programmed according to the teachings of the present disclosure, as will be apparent to those skilled in the computer art. Appropriate software coding can readily be prepared by skilled programmers based on the teachings of the present disclosure, as will be apparent to those skilled in the software art. The invention may also be implemented by the preparation of integrated circuits or by interconnecting an appropriate network of conventional component circuits, as will be readily apparent to those skilled in the art.

The foregoing description of various embodiments of the claimed subject matter has been provided for the purposes of illustration and description. It is not intended to be exhaustive or to limit the claimed subject matter to the precise forms disclosed. Many modifications and variations will be apparent to the practitioner skilled in the art. Particularly, while the concept “component” is used in the embodiments of the systems and methods described above, it will be evident that such concept can be interchangeably used with equivalent concepts such as, class, method, type, interface, module, object model, and other suitable concepts. Embodiments were chosen and described in order to best describe the principles of the invention and its practical application, thereby enabling others skilled in the relevant art to understand the claimed subject matter, the various embodiments and with various modifications that are suited to the particular use contemplated.

What is claimed is:

1. A system to support a breath monitoring mattress for sleeping disorder detection, comprising:
one or more breathing sensors embedded in the mattress and configured to
detect body movements and/or breathing activities of a person occupying the mattress;
provide electronic data of the person's body movements and/or breathing activities detected to a breathing monitor for analysis;
said breathing monitor configured to
identify a breathing pattern of the person on the mattress based on analysis of the data of the person's body movements and/or breathing activities received from the breathing sensors;
detect a sudden or abnormal change in the person's breathing pattern when such change happens, wherein such sudden or abnormal change is an indication that the person is experiencing a sleeping disorder;
albert the person and/or his/her caregiver to take action once the sudden or abnormal change in the person's breathing pattern is detected.

2. The system of claim 1, wherein:
the mattress is a crib-sized mattress for infant or toddler, wherein the system is configured to prevent the infant or toddler sleeping on the mattress from suffering sudden infant death syndrome (SIDS) or infant sleep apnea.

3. The system of claim 1, wherein:
the breathing sensors are positioned within or underneath a top layer of the mattress in proximity to the person who occupying the mattress, wherein the breathing sensors are configured to detect a change or distortion in shape or contour of the top layer of the mattress caused by the body movements and/or breathing activities of the person.

4. The system of claim 1, wherein:
the breathing sensors are coupled to one or more coil springs inside the mattress, wherein the breathing sensors are configured to detect suppression of the coil springs as a result of the body movements and/or breathing activities of the person.

5. The system of claim 1, wherein:
the breathing sensors are configured to communicate with the breathing monitor via a wired connection between the breathing and the breathing monitor.

6. The system of claim 1, wherein:
the breathing sensors are configured to communicate with the breathing monitor via a wireless communication network.

7. The system of claim 1, wherein:
the breathing monitor is a dedicated electronic hardware device that includes a display screen and one or more input devices, control buttons, and LCD indicators.

8. The system of claim 1, wherein:
the breathing monitor is a mobile computing/communication device having a breath monitoring software running on it.

9. The system of claim 1, wherein:
the identified breathing pattern includes one or more of frequency of the person's breath, duration and depth of each breath.

10. The system of claim 1, wherein:
the breathing monitor is configured to determine a current resting state of the person on the mattress based occupying the breathing pattern of the person, wherein the resting state is one of awake state, light sleeping state, and deep sleeping state.

11. The system of claim 1, wherein:
the breathing monitor is configured to automatically trigger an alarm sound through a speaker associated with or integrated within either the breathing monitor or the mattress to wake the person up.

12. The system of claim 1, wherein:
the breathing monitor is configured to automatically trigger a vibrator embedded in the top layer of the mattress to wake the person up.

13. The system of claim 1, further comprising:
a monitoring device associated with the caregiver of the person and configured to receive information about the person's breathing pattern and the sudden or abnormal change detected in the breathing pattern from the breathing monitor, wherein the caregiver is at a separate location from the breathing monitor and/or the person sleeping on the mattress.

14. The system of claim 13, wherein:
the monitoring device is configured to present the information about the person's breathing pattern to the caregiver via a display and to notify or alert the caregiver by sound or vibration when the sudden or abnormal change in the breathing pattern of the person is detected.

15. A method to support a breath monitoring mattress for sleeping disorder detection, comprising:
detecting body movements and/or breathing activities of a person occupying the mattress via one or more breathing sensors embedded in the mattress;
providing electronic data of the person's body movements and/or breathing activities detected to a breathing monitor for analysis;
identifying a breathing pattern of the person on the mattress based on analysis of the data of the person's body movements and/or breathing activities received from the breathing sensors;
detect a sudden or abnormal change in the person's breathing pattern when such change happens, wherein such sudden or abnormal change is an indication that the person is experiencing a sleeping disorder;
albert the person and/or his/her caregiver to take action once the sudden or abnormal change in the person's breathing pattern is detected.

16. The method of claim 15, wherein:
the breathing sensors are positioned within or underneath a top layer of the mattress in proximity to the person who occupying the mattress or coupled to one or more coil springs inside the mattress.

17. The method of claim 15, further comprising:
determining a current resting state of the person on the mattress based occupying the breathing pattern of the person, wherein the resting state is one of awake state, light sleeping state, and deep sleeping state.

18. The method of claim 15, further comprising:
automatically triggering an alarm sound through a speaker associated with or integrated within either the breathing monitor or the mattress to wake the person up.

19. The method of claim 15, further comprising:
automatically triggering a vibrator embedded in the top layer of the mattress to wake the person up.

20. The method of claim 19, further comprising:
receiving information about the person's breathing pattern and the sudden or abnormal change detected in the breathing pattern via a monitoring device associated with the caregiver of the person, wherein the caregiver is at a separate location from the breathing monitor and/or the person sleeping on the mattress.
